(19) United States
(12) Patent Application Publication
(10) Pub. No.: US 2020/0214675 A1
(43) Pub. Date: Jul. 9, 2020

(54) 3D ULTRASOUND SYSTEM AND METHOD

(71) Applicant: Her Majesty The Queen in Right of Canada, as Represented by The Minister of National Defence, Ottawa (CA)

(72) Inventor: Stergios STERGIOPOULOS, North York (CA)

(21) Appl. No.: 16/708,869

(22) Filed: Dec. 10, 2019

Related U.S. Application Data
(60) Provisional application No. 62/777,321, filed on Dec. 10, 2018.

(51) Int. Cl. A61B 8/08; A61B 8/00

(52) U.S. Cl. CPC A61B 8/483; A61B 8/4494; A61B 8/466

(57) ABSTRACT

Ultrasound imaging is performed in three dimensions by firing a multidimensional array of ultrasound transducers comprising N transducers at a target. First reflected signals are sensed with at least some of the N transducers to produce first output signals. The first output signals are provided from only a first portion of the N transducers less than all the N transducers for storage and adaptive beamforming. The multidimensional array of ultrasound transducers comprising N transducers is fired at the target another time to produce second reflected signals. The second reflected signals are sensed with at least some of the N transducers to produce second output signals. The second output signals are provided from only a second portion of the N transducers less than all the N transducers and different from the first portion for storage and adaptive beamforming.

### FIELD OF THE INVENTION

The invention relates to the field of imaging and more particularly to a novel architecture and method for capturing data for forming a 3D ultrasound dataset.

### BACKGROUND

Approximately 90% of trauma deaths occur in an accident zone prior to medical or surgical interventions. This used to be the obvious result of untreatable massive injury, but advances in medicine now allow a lot of traumatic injuries to be treated when diagnosed and addressed early enough.

Unfortunately, the lack of intelligent diagnostic tools that are capable of providing rapid and accurate diagnosis of non-visible internal injuries is the major challenge facing medical personnel, especially in mass casualty situations, under-served regions, and far forward operations within the defense sector. To this day, there is no portable system that provides relevant image data and automated diagnostic tools for use at the site of a trauma. Such a system with minimal training requirements should be capable of detecting life threatening injuries within the so-called “golden hour” of trauma diagnosis. The stress, commotion and the non-specific signs—symptoms—of trauma and the variability of patient reactions to injury result in frequently unreliable physical examinations in trauma settings. This in turn has been known to lead to catastrophic results.

Ultrasounds have been widely used for medical diagnosis in hospital and doctor's offices. “Pre-hospital” ultrasound has been used in emergency ambulances and helicopters mainly in North America and Central Europe since the late 1990s. The use of “in field” ultrasound has also been considered for mass casualty incidents. In all cases its use is restricted to specialists or well-trained staff, which are not available in most emergency crews. Although ultrasound systems with a high degree of mobility—smart-phone-sized, PDA based-systems, e.g. VSCAN or Signos—are available, these systems have limited diagnostic utility, offer no automated diagnosis options, and cannot be used reliably by first responders. In practice, experienced first respondents are using their long-term experience and knowledge during triage. More specifically, to find a relevant internal anatomical region using 2D ultrasound imaging, placement of an ultrasonic probe and the assessment of a 2D image—B-scan image—requires training and long-term experience. This is simply not possible in typical paramedic situations.

As well as for a civilian emergency care market, both the US and Canadian Armed Forces desire a field-deployable compact 4D (3D+Time) ultrasound imaging system capable of providing rapid diagnosis and triage of non-visible internal injuries. The need for such a system has been identified by both the Canadian Forces Surgeon General and the US-Army Director of the Combat Casualty Care Program. NATO allies, U.K. and Germany have issued similar requirements. Unfortunately, a portable and field operable real time 3D ultrasound imaging system is unavailable.

It would be advantageous to provide a portable trauma detection system for use by first responders.

### SUMMARY

In accordance with the invention there is provided a method comprising: firing a multidimensional array of ultrasound transducers comprising N transducers at a target; sensing first reflected signals with at least some of the N transducers to produce first output signals; providing the first output signals from only a first portion of the N transducers less than all the N transducers for storage and adaptive beamforming; firing the multidimensional array of ultrasound transducers comprising N transducers at the target another time to produce second reflected signals; sensing the second reflected signals with at least some of the N transducers to produce second output signals; and providing the second output signals from only a second portion of the N transducers less than all the N transducers and different from the first portion for storage and adaptive beamforming.

In some embodiments, the first reflected signals are digitized to form the first output signals and wherein the second reflected signals are digitized to form the second output signals.

In some embodiments, the method comprises: performing adaptive beamforming on data derived from the first output signals and from the second output signals to determine a sensed topography; and displaying an image of the sensed topography.

In some embodiments, the method comprises: firing the multidimensional array of ultrasound transducers comprising N transducers at the target another time; sensing third reflected signals with at least some of the N transducers to produce third output signals; and providing the third output signals from only a third portion of the N transducers less than all the N transducers and different from the first portion and different from the second portion for storage and adaptive beamforming.

In some embodiments, the first reflected signals are digitized to form the first output signals, wherein the second reflected signals are digitized to form the second output signals, and wherein the third reflected signals are digitized to form the third output signals.

In some embodiments, the method comprises: performing adaptive beamforming on data derived from the first output signals, from the second output signals, and from the third output signals to determine a sensed topography; and displaying an image of the sensed topography.

In some embodiments, firing the multidimensional array of ultrasound transducers comprises forming a same illumination pattern with the multidimensional array of ultrasound transducers each time.

In some embodiments, the illumination pattern is conical.

In some embodiments, the illumination pattern is formed by beam steering and wherein each of the output signals used in adaptive beam forming for same output image data are captured relying upon a same illumination pattern.

In some embodiments, groups of the multidimensional array of ultrasound transducers are coupled via a multiplexer and addressed simultaneously, the multiplexing allowing the entire multidimensional array of ultrasound transducers to be read in n successive operations by incrementing the multiplexer addressing between operations.

In some embodiments, each multiplexer addresses four different ultrasound transducer elements of the multidimensional array of ultrasound transducers.

In some embodiments, adaptive beam forming is performed on an image comprising data from all of the transducer elements within the multidimensional array of ultrasound transducers.

In some embodiments, adaptive beamforming is performed in reliance upon two previously captured images, each of the previously captured images captured relying on a same illumination pattern.

In some embodiments, adaptive beam forming is performed in the frequency domain.

In accordance with the invention there is provided an ultrasound system comprising: a multidimensional transducer array comprising a plurality of ultrasound transducer elements arranged in an array, each of the plurality N of ultrasound transducers arranged for transmitting a beam steered signal together, and each of the plurality of transducers coupled to a multiplexer for switching between n of the plurality of ultrasound transducers such that there are at least N/n mulitplexers; the multiplexers coupled for providing an information output signal from a selected one of the n transducers coupled therewith in response to a selection signal, each multiplexer for selecting between n transducers coupled therewith such that sampling of the information output signal from the N transducers is performed in n operations.

In some embodiments, each of the plurality of ultrasound transducers is arranged for transmitting a steered signal simultaneously and only N/n ultrasound transducers are for being read simultaneously.

In some embodiments, the multiplexers are each a 4:1 multiplexer.

In some embodiments, the system comprises an analog to digital converter coupled to an output port of each of the at least N/n mulitplexers.

In accordance with the invention there is provided a method comprising: providing a multidimensional array of ultrasound transducers comprising a plurality of ultrasound transducer elements for transmitting ultrasound signals and for sensing reflected signals, the ultrasound transducer elements addressable in parallel for providing transmit signals to support beam steering and addressable in at least three sets of ultrasound transducer elements fewer than all the ultrasound transducer elements for transferring received reflected signal information therefrom; providing at least a signal for forming a steered beam transmitted from all of the ultrasound transducer elements within the array; and providing an address for addressing one set of the at least three sets to read received signal information therefrom.

In accordance with the invention there is provided a method comprising: firing a multidimensional array of ultrasound transducers comprising N transducers at a target; sensing first reflected signals with at least some of the N transducers to produce first output signals; digitizing the first output signals to produce digitized first output signals; transmitting some of the first output signals to a first processing circuit for performing Csteer processing; transmitting others of the first output signals to a second other processing circuit for performing Csteer processing; transmitting an output signal from the Csteer from each of the first processing circuit and the second processing circuit to a third processor for performing Rsteer processing; and transmitting an output signal from the Csteer from each of the first processing circuit and the second processing circuit to a fourth other processor for performing Rsteer processing.

In accordance with the invention there is provided a method comprising: capturing first received ultrasound image information from a first set of ultrasound transducer elements less than all the ultrasound transducer elements within a multidimensional ultrasound transducer array; constructing a first superset comprising information from each of the ultrasound transducer elements within the multidimensional ultrasound transducer array and including the captured first received ultrasound image information, some of the information captured previously; performing adaptive beamforming on the first superset; capturing second received ultrasound image information from a second set of ultrasound transducer elements less than all the ultrasound transducer elements within a multidimensional ultrasound transducer array and different from the first set; constructing a second superset comprising information from each of the ultrasound transducer elements within a multidimensional ultrasound transducer array including the second received ultrasound image information, some of the information captured previously; and performing adaptive beamforming on the second superset.

In accordance with yet another embodiment of the invention there is provided an ultrasound system comprising: a plurality of ultrasound transducers arranged in an array, each of the plurality of ultrasound transducers arranged for transmitting a signal together, and a first group of the plurality of ultrasound transducers arranged for receiving a reflected signal at a first time and a second group of the plurality of ultrasound transducers arranged for receiving a reflected signal at a second time, the first group different from the second group; a multiplexer for switching between the first group of the plurality of ultrasound transducers and the second group of the plurality of ultrasound transducers for providing information therefrom to a digitizing circuit for providing digitized sensed data; a processor configured for: assembling the digitized sensed information from the first group of the plurality of ultrasound transducers and the digitized sensed information from the second group of the plurality of ultrasound transducers to form a digitized sensed data set; processing the digitized sensed data set by performing adaptive beamforming on the digitized sensed data set to provide image data; and displaying the image data.

In some embodiments, displaying the image data is performed in real time.

In accordance with yet another embodiment of the invention there is provided a method comprising: firing an array of sensors comprising N sensors at a target; sensing reflected signals with at least some of the N sensors; digitizing the first output of at least some of the N sensors; converting the digitized first output from each of the at least some sensors into the frequency domain; providing the converted digital output from only a first portion of the N sensors less than all the N sensors for storage and adaptive beamforming; firing the array of sensors comprising N sensors at the target; sensing reflected signals with at least others of the N sensors; digitizing the second output of at least the others of the N sensors; providing the digital output from only a second portion of the N sensors less than all the N sensors and different from the first portion for storage and adaptive beamforming; performing adaptive beamforming on the data; and displaying an image of the sensed topography within the sensors range.

In some embodiments, displaying the image data is performed in real time.

### BRIEF DESCRIPTION OF THE DRAWINGS

Exemplary embodiments of the invention will now be described in conjunction with the following drawings, wherein similar reference numerals denote similar elements throughout the several views, in which:

FIG. 1 is a simplified diagram of a model for physical actuator based ultrasound scanning;

FIG. 2 shows a simplified two dimensional array representative of a two dimensional array transducer that is divided into sets of 4 cells, 2×2, allowing for multiplexer addressing of one quarter of the array elements at a time;

FIG. 3 is a simplified architectural diagram of a connector board supporting 32 channels—128 channels when addressed sequentially for four image capture operations;

FIG. 4 is a simplified diagram of a housing including a sensor, processing boards, and interconnects in the form of cabling;

FIG. 5 is a diagram showing illumination patterns achievable with a single firing in some embodiments of the invention;

FIG. 6 is a group of images formed with a four-dimensional (3 spatial and time) ultrasound showing different anatomical features of interest;

FIG. 7(a) is a simplified schematic diagram of an image capture system for capturing a digital image relying on n separate image capture operations to form a single image;

FIG. 7(b) is a simplified schematic diagram of a processing system for processing image data within the frequency domain;

FIG. 7(c) is a simplified schematic diagram of a plurality of processing systems shown in FIG. 7(b) operating in parallel;

FIG. 7(d) is a simplified timing diagram for processing of captured image data;

FIG. 8 is a simplified architectural diagram showing data connections between different components of a parallel architecture ultrasound processing system;

FIG. 9 is a diagram of a cylindrical array of ultrasound transducers; and

FIGS. 10a and 10b form a simplified flow diagram for mathematically processing of ultrasound data.

### DETAILED DESCRIPTION OF EMBODIMENTS

The following description is presented to enable a person skilled in the art to make and use the invention and is provided in the context of a particular application and its requirements. Various modifications to the disclosed embodiments will be readily apparent to those skilled in the art, and the general principles defined herein may be applied to other embodiments and applications without departing from the scope of the invention. Thus, the present invention is not intended to be limited to the embodiments disclosed but is to be accorded the widest scope consistent with the principles and features disclosed herein.

### DEFINITIONS

Object under test (OUT): An object under test is any object, objects, and/or body that is being imaged using ultrasound imaging techniques.

Two-dimensional (2D): Two-dimensional implies that a result is traversable along each of two separate dimensions. Many different two-dimensional coordinate systems are known including Cartesian co-ordinates, radial co-ordinates, etc.

Three-dimensional (3D): Three-dimensional implies that a result is traversable along each of three separate dimensions. Many different three-dimensional coordinate systems are known including Cartesian co-ordinates (along each of three orthogonal vectors), cylindrical co-ordinates, conical coordinates, etc.

Four-dimensional (4D): Four-dimensional implies that a result is traversable along each of three separate dimensions and time, providing a three-dimensional image for each of a series of instances in time.

No portable ultrasound systems are presently available that provide three-dimensional imaging in sequence, thereby supporting four dimensions—three special dimensions and time. Further, there is no system that addresses portable, self-contained, rugged, field operable equipment for civilian needs and also for military needs at a front line of combat operations.

Available portable ultrasound systems provide two-dimensional images. Unfortunately, two-dimensional images are quite limited in their representation of internal scanned objects and, as such, require a trained medical professional to mentally integrate multiple images to develop a three-dimensional impression of internal scanned objects. Not only does two-dimensional ultrasound imaging require significant training to be useful, but it is also time-consuming and inefficient, making it very poorly suited to use in war zones and at the scene of traumatic events. Making a system that involves less human concentration and less time is important for triage type applications. Making a system that requires less training is important for wider adoption and deployment, as well as for versatility and scalability of system utilization.

Problematically, prior art solutions to 3D volumetric imaging are slow and require extensive hardware that is not portable in nature. The resulting systems are ill suited to mobile applications such as for use in triage situations, including those in both civilian settings and in military combat situations. Further, miniaturization of these systems if it is possible would only make them slower and even more poorly suited to battlefield use.

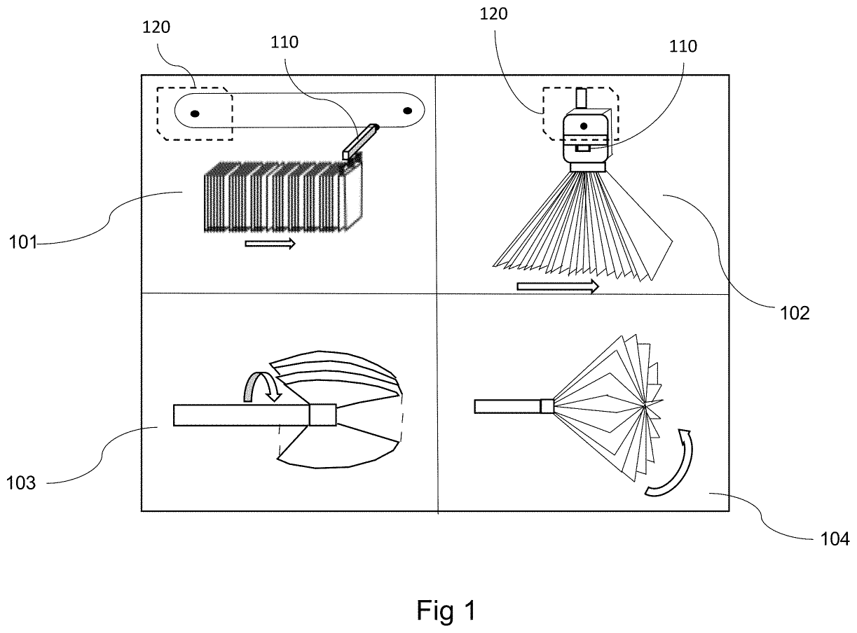

Three-dimensional ultrasound imaging systems have three components: an image acquisition circuit, a data reconstruction circuit for constructing a three-dimensional or four dimensional image, and a display circuit for displaying the resulting image data. The three-dimensional image reconstruction process is achievable by a mechanical scanning technique as depicted in FIG. 1. In other solutions, a matrix planar array ultrasound probe is used.

For mechanical scanning, a linear ultrasound transducer 110 is mounted for being moved by a motor 120 as shown in the top two images 101 and 102 of FIG. 1. The motor moves the linear array, either laterally across the object under test as shown image 101 or angularly as shown in image 102 to capture the object under test from a single scanning location in a sweeping fashion. The movement of the ultrasound transducer is continuous, cardiac and/or respirator. In addition, the spatial-sampling frequency of the image acquisition is adjustable based on the elevational resolution of the transducer and the depth of the region-of-interest. Thus, for linear scanning, 140 images with a resolution of 336×352 pixels each are collected at 0.5 mm spatial intervals with a time interval dependent upon the ultrasound system frame rate and whether cardiac gating is used. Typically, scanning parameters are adjustable based on requirements of a given experiment or application. For example, for three-dimensional B-mode—typically 2 or 3 focal zones are used resulting in a capture rate of approximately 15 frames/sec and a total three-dimensional imaging scanning time of 9 seconds for 140 ultrasound transducer images. Although this technique does produce useful three-dimensional image data, it suffers from major limitations that preclude its use for general diagnostic procedures. Notably, manual scanning of the three-dimensional space with a linear array does nothing to eliminate false components within the reconstructed B-scan images that are inherent in B-scans.

Three-dimensional image acquisition processes for matrix planar array ultrasound probes, are technically more challenging. Deployed planar arrays include a large number of transducers, typically in the range of 64×64=4096 elements; however, processing and architectural limitations result in the number of elements used being approximately 8×8=64 for a 3D beamformer. Thus, the array gain of a small size sub-array of typically 8×8=64 elements is reduced by approximately: 10×Log₁₀(64)=18 dBs when compared to the full array gain that would have been provided by the full planar array of 64×64=4096 elements, had it been usable.

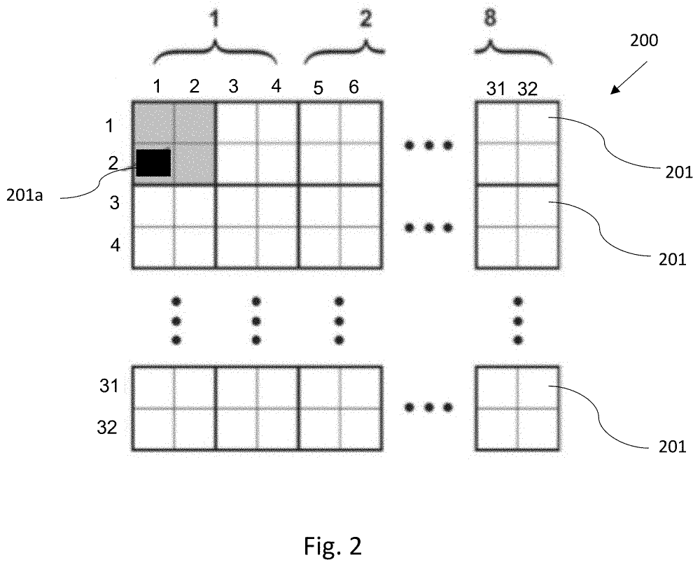

For the sake of simplicity and without any loss of generality, the three-dimensional ultrasound beamformer coherently processes a received signal of only 8×8=64-elements or 4×4=16 elements, which is a sub-aperture of the 64×64=4096-elements within the β planar array shown at 200 in FIG. 2. Active transmission takes place approximately every 0.3 ms, depending on desired penetration depth in the object under test. This is much faster than the 4 seconds taken for each image when mechanical scanning is being used.

When an active transmission is completed, the receiving 8×8=64-element sub-aperture is shifted to the left or right by a few elements. Thus, to make use of all the 4096-elements of the deployed probe, the 8×8=64-element beamforming process is repeated at least 32 to 64 times, generating numerous beams at numerous times. For a 4×4=16 sub-aperture, there is at least 4 times as many beamforming processes over the 8×8 aperture resulting in 128-256 imaging operations.

As a result, resulting angular resolution characteristics of reconstructed image data are defined by array gain of the three-dimensional beamformer of the 8×8=64 element sub-aperture as opposed to by the 64×64=4096-element planar array.

The current solution does not support a mobile, light-weight, easy-to-use, easy-to-operate, trauma detection and assessment system. Instead, it requires significant hardware and complexity and due to its complexity and performance is ill suited to battlefield and triage applications.

By recording a three dimensional volumetric region with deep penetration (i.e. 24 cm) and wide 3D angle coverage 80°×80°, relevant anatomical structures of interest will be included in the volumetric image and therefore, convenience in locating an anatomical structure and a probability that such a structure is missed by a paramedic with knowledge of human anatomy, will be very low. Moreover, by enhancing image quality and including a temporal component in the sample volume—a time line of successive images, a rapid diagnostic assessment in remote areas becomes more reliable and robust, even without specialized training in system operation.

Alternatively, as shown in FIGS. 1 at 103 and 104, a rotation of a linear array is used to illuminate and capture image data relating to a three-dimensional volume.

In the present embodiment, the following elements are combined to provide a 4D digital ultrasound system.

A two-dimensional Matrix Array Probe forms part of the present embodiment. Though this is a challenging development, there are transducers available such as from Fraunhofer-IBMT (St. Ingbert, Germany), which has built a 32×32 transducer 2D planar array probe that has been integrated with an experimental prototype, detailed hereinbelow. Vermon, in France, has also built a multi-dimensional planar array with 32×32 elements. However, a 16×16 or 64×64 planar array is also suitable to the embodiment.

Preferably, the planar array incorporates the following structures in order to result in substantial miniaturization and improved simplicity of the remaining system architecture.

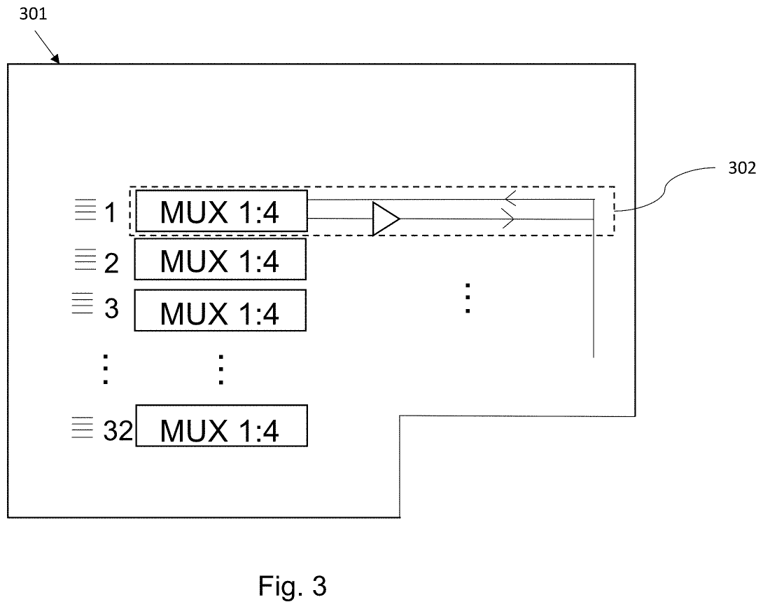

A multiplexer (MUX) for multiplexing planar array elements within the probe. In the preferred embodiment, as shown in FIG. 2, a 4 element MUX—2×2—(not shown) is used to select between four neighboring array elements 201, as shown at (1:1) to (2:2), the four elements forming a single switching group. Each array element comprises a piezoelectric element 201a within a same array and implemented on one connector board designated by numerals 1, 2, . . . 8 as shown in FIG. 3. Thus, though the array of FIG. 2 comprises 32×32 elements, only 16×16 elements are addressed for reading simultaneously via the 4:1 MUX's.

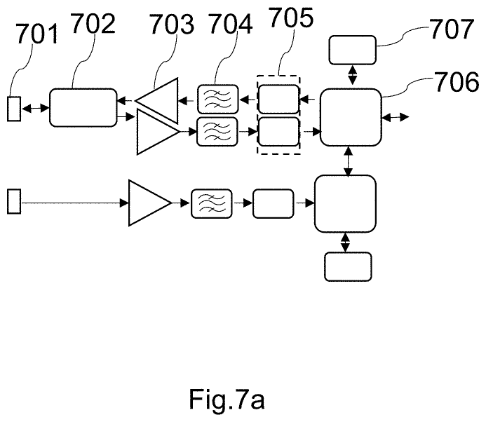

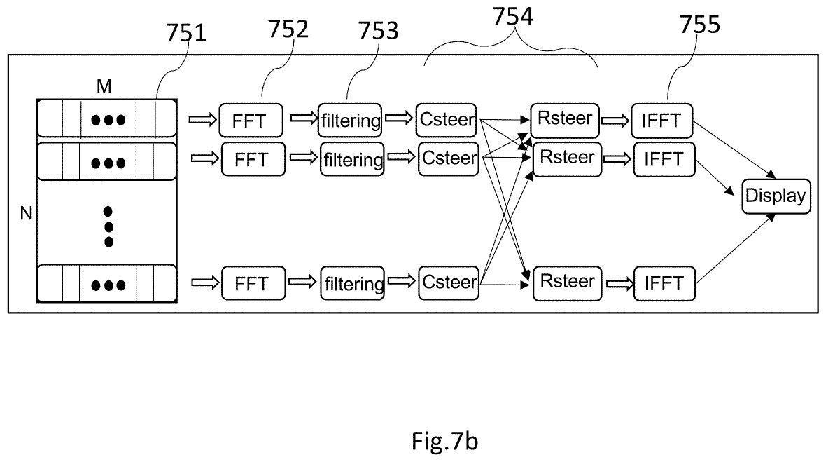

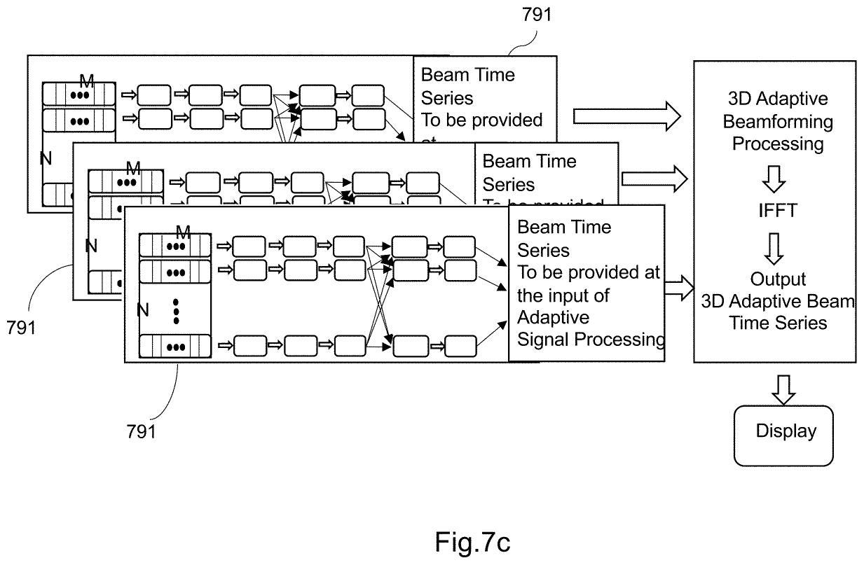

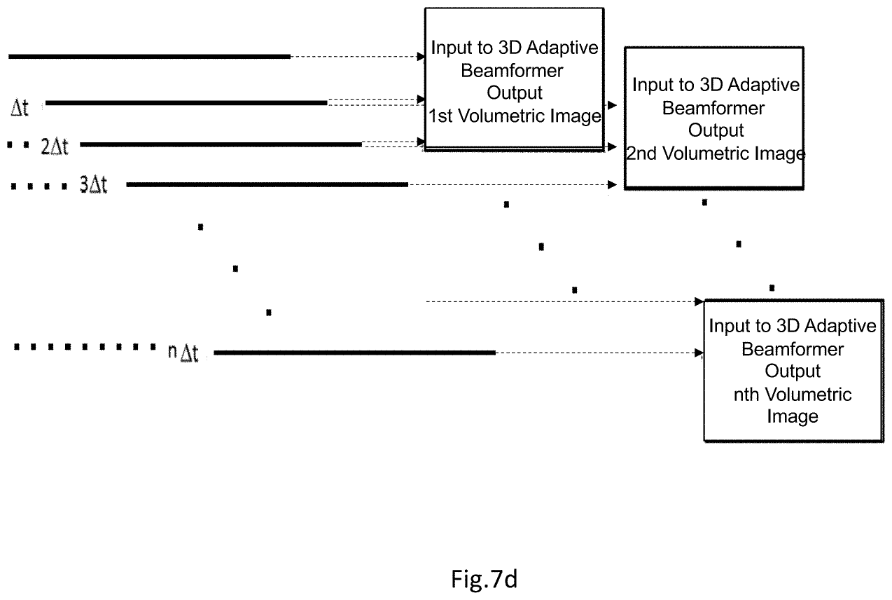

In an exemplary embodiment with a 32×32=1024 planar array, 1024 Analog to Digital Conversion (ADC) operations are performed. Because of the inclusion of MUX for addressing transducers, the ADC operations are performed in four (4) sequential sets of operations thereby relying upon 256 Analog to Digital Conversion (ADC) circuits with each circuit performing 4 Analog to digital conversions in series—one after another. In FIG. 3, a simplified bus diagram for a connector board 301 having 32 multiplexer and Analog to Digital Conversion (ADC) channels 302, thereby supporting 128 channels is shown; 8 such boards are used to support the total number of the 1024 channels of the 32×32 planar array with 4 successive operations of 256 channels. Referring to FIG. 7(a), shown is a simplified block diagram of each channel. A transducer group 701 is coupled via a MUX 702 to the connector board. On the connector board is an amplification circuit 703 for performing pre-amplification of received signals, a filter 704, and analog/digital conversion circuitry 705. A processing circuit in the form of a processor implemented within programmable logic 706 interfaces between the analog/digital conversation circuitry 705 and a memory circuit 707, providing data storage management and processing for the system. In an exemplary embodiment, a maximum transmit voltage of 80 V peak-peak is supported. Alternatively, another number of ADC circuits is supported on each connector board.

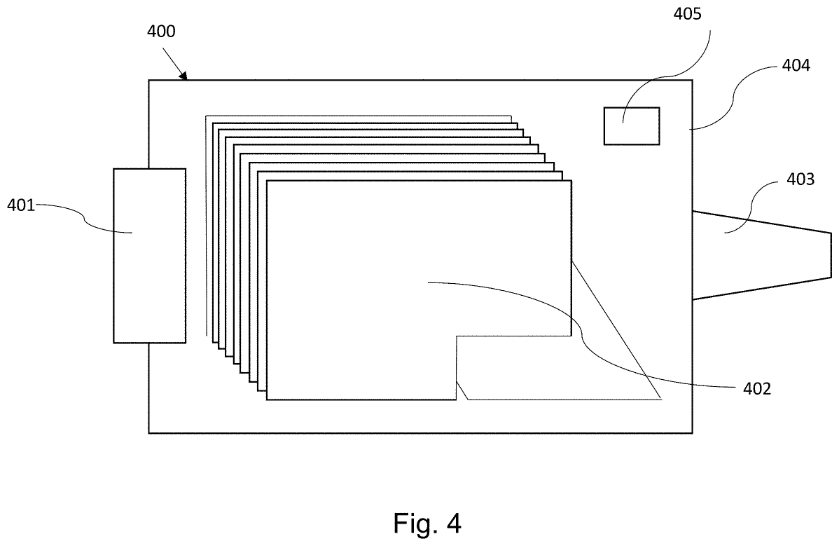

The four (4) 16×16 (256) groups of transducers 701 coupled to each MUX 702, are capable of active transmission of digital active signals through 256-channels of Digital to Analog Conversion (DAC) and reception of acoustic signals through 256-channels of ADC. As a result, pre-amplification functionality provides a protection for the active elements to minimize interference for the remaining reception channels of the matrix array. Referring to FIG. 4, the probe in the form of the sensor head 400, including the planar matrix array 401, is packaged to include connection of the planar matrix array 401 with 8 connector boards 402, backplane (not shown), cabling 403 and housing 404. Thus, the sensor and electronics are housed within a same sensor head 400. A thermal trap or cooling 405 should be included if necessary, as shown in FIG. 4. Alternatively, the sensor is housed in the sensor head 400 and the remaining electronics is housed within a control portion of the system. When this is the case, care should be taken to avoid the effects of noise for each of the 256 channels.

Though the exemplary embodiment is described with reference to 8 connector boards, and a 4:1 MUX, other configurations are also applicable and optionally depend on the geometry of the planar array. For example, if an 8:1 MUX is used then only 4 connector boards as described hereinabove are connected. Alternatively, each connector board supports only half as many channels and a same number of connector boards are used. Further, a radial array may be best served by a different ratio of multiplexer.

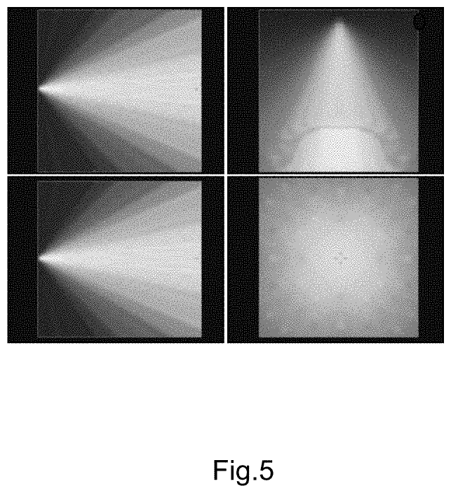

Referring to FIG. 5, the illumination patterns that have been tested, comprise a set of multi-focus, multi-angular sectors that are illuminated simultaneously by a single firing. For comparison, a current ultrasound system utilizes at least 256 firings to illuminate a single plane. The illumination patterns depicted in FIG. 5 simplify an illumination process. This is achieved in the present embodiment through a fully digital design configuration of the illumination driver architecture. Alternatively, another illumination pattern is used, or multiple illumination patters are used within a same system.

The resulting illumination from the above matrix planar array structure is as follows: A conical volumetric segment is imaged with an opening angle of 80 degrees×80 degrees to a maximum depth of 24 cm (sample volume) with an angular resolution of 0.5 degrees and a rate of 20 volumes per second (Vps). A 2D-phased array probe with 32×32 single elements working at a center frequency of 3.0 MHz is used; the frequency is based on the probe design and with some probes, frequencies such as 7-9MHz are used. All 1024 elements are active during the transmit phase, forming an illumination pattern such as that shown in FIG. 5. During the receive phase, a group of 256 elements are addressed—connected to the 256 electronic channels of the system—by way of addressing the multiplexers. For this embodiment, all multiplexers are coupled to identical addressing circuitry. Alternatively, each multiplexer has its own addressing circuitry and each multiplexer is addressed same during use. Alternatively, the ultrasound signal is designed with different maximum depth and/or different opening angles.

Four receive operations are necessary to acquire ultrasound responses from a whole volume of interest with all 1024 transducer elements. This is done with 4 transmit operations—transmitting from all transducer elements—each followed by a receive operation—each for receiving information from ¼ of the transducers—wherein addressing of the MUX circuits is incremented between operations. Alternatively, another order of addressing the MUX circuits is also supported.

Switching of the elements between transmit and receive phase and the correct choice of the relevant receive elements is done by addressing and operation of the multiplexers shown in FIGS. 2-4 that are integrated into the probe. The 256 received A-Scans of one shot are digitized with a sampling rate of 25 MHz (or higher), a 14 bit (or 12-bit) resolution, and a length of 4096 samples. After four shots, the 1024 A-Scans of one sample volume are processed by filtering, adaptive beamforming, and scan conversion. The processed A-Scans are then transmitted for display on a monitor, where they are displayed in a 3D representation. This entire process is performed 20 times per second so that a quasi-real-time 4D imaging is achieved. Alternatively, each A-scan results in processing of the A-scan and three previous A-scans to allow for 80 frames per second of quasi-real-time 4D imaging.

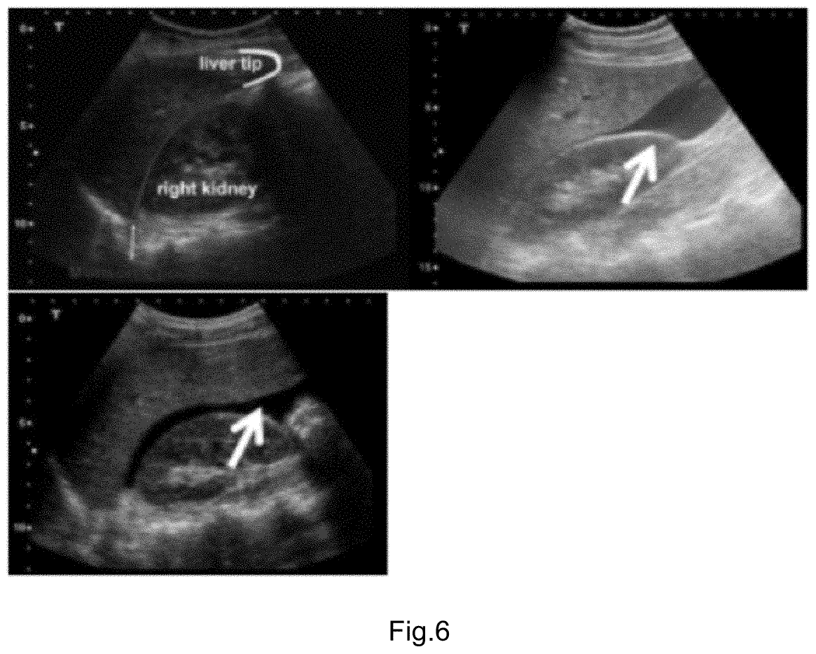

On a monitor is shown an image and a graphical user interface. In some embodiments, a result of automated detection of free fluid—intraperitoneal free fluid or blood—inside the sample volume is also shown as shown in FIG. 6. In an exemplary embodiment, automated diagnosis is integrated, but is restricted to detecting free fluid in a characteristic region called Morrison-Pouch, which is the space that separates the liver from the right kidney shown in FIG. 6 and indicated by an arrow. When a person is lying on their back, this is the deepest region inside the abdominal cavity—a region where fluid collects in the case of an internal injury with bleeding.

In U.S. Pat. Nos. 6,719,696 & 6,482,160, a 3D adaptive beamforming method is disclosed. Each of the U.S. Pat. Nos. 6,719,696 & 6,482,160 are incorporated herein by reference. The references taken together define the signal processing structure of an adaptive multidimensional beamformer having instantaneous convergence for ultrasound imaging systems deploying multidimensional sensor arrays. The method provides for enhanced angular resolution of the resulting beamformer. Unfortunately, the method and system are complex and are not amenable to portability and mobile application.

An explanation of a method of digital adaptive beamforming is now presented.

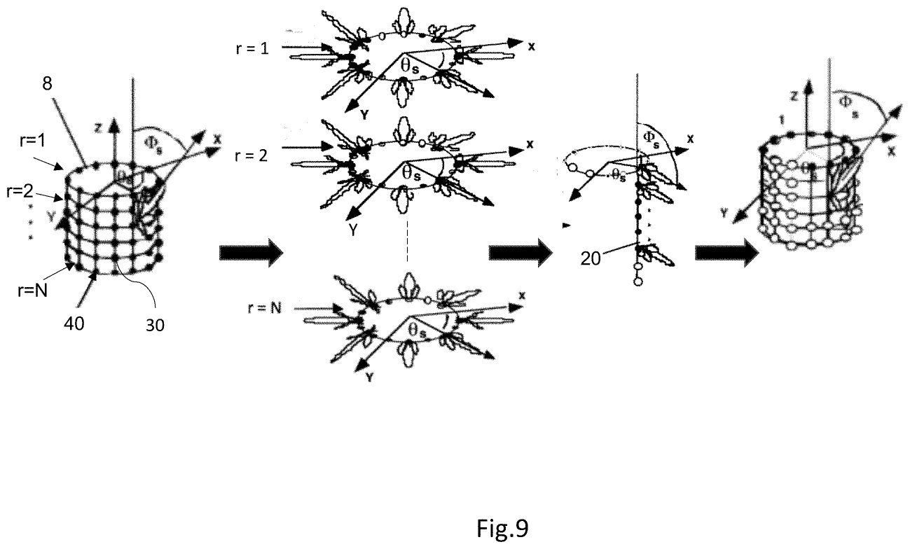

Conventional (Time Delay) 3D Beamformers in Frequency Domain:
The first step in the implementation of a multidimensional adaptive beamformer in a multidimensional ultrasound transducer array probe is defined by a decomposition process as described hereinbelow for a cylindrical array beamformer, but the process is similar for planar arrays and is extensible thereto. Decomposition of a cylindrical array beamformer into coherent sub-sets of linear and circular array beamformers is described. A cylindrical sensor array 40 as shown in FIG. 9 comprises NM sensors 8, with N being the number of circular sensor arrays 30 that are disposed in a column and M being the number of sensors 8 on each circular sensor array 30 that are disposed radially about each circular sensor array 30, that is the number of line sensor arrays 20. Angular response of the cylindrical sensor array 40 to a steered direction at (θₛφₛ) as indicated in FIG. 9 is expressed as follows (equation 3):

$$B ( f , {θ}_{s} , {φ}_{s} ) = {\sum }_{r = 0}^{N - 1} {\sum }_{m = 0}^{M - 1} {w}_{r , m} {X}_{r , m} ( f ) {d}_{r , m}^{*} ( f , {θ}_{s} , {φ}_{s} ) . \qquad ( 3 )$$

W_{r,m} is the (r,m)^{th} term of matrix W(θ,φ) comprising weights of a three dimensional spatial window to suppress sidelobe structures. X_{r,m}(f) is the (r,m)^{th} term of matrix X(f) expressing the Fourier Transform of a sensor time series of the m^{th} sensor on the r^{th} circular array. D(f,θₛ,φₛis a steering matrix having its (r,m)^{th} phase term for a plane wave signal expressed by dr,m(f,θₛ,φₛ)=exp(j2πf(rδ_{z} cos φₛ+R sin φₛcos (θₛ θₘ))/c), with R being a radius of the circular sensor array 30, δz being a distance between subsequent circular arrays in z-direction and θₘ=2πm/M, m=0,1, . . . , M 1 indicating a position of a sensor 8 on the circular array 30. A re-arranged form of equation (3), is expressed as follows:

$$B ( f , {θ}_{s} , {φ}_{s} ) = {\sum }_{r = 0}^{N - 1} {w}_{1 , r} {d}_{r}^{*} ( f , {θ}_{s} , {φ}_{s} ) [ {\sum }_{m = 0}^{M - 1} {X}_{r , m} ( f ) {w}_{2 , m} {d}_{m}^{*} ( f , {θ}_{s} , {φ}_{s} ) ] . \qquad ( 4 )$$

dᵣ(f,θₛφₛ)=exp(j2πfrδ_{z} cos φₛ/c) is the r^{th} term of steering vector D̅z(f,θ,φs), which is the kernel of a line array beamformer as discussed above and w1,r is the term of a spatial window for line array spatial shading. dₘ(f,θₛ,φₛ)=exp(j2πfR sin φₛ cos(θₛ θₘ)/c) is the m^{th} term of steering vector D̅y(f,θs,φs), which is the kernel of a circular array beamformer as discussed above and W_{2,m} is the term of a spatial window for circular array spatial shading. Equation (4) expresses a cylindrical array beamformer comprising a product of two summations, the first being a linear array beamformer and the second being a circular array beamformer. Accordingly, a beamforming process for a cylindrical array as expressed by equation (4) is decomposed into two steps as shown in FIG. 9. In a first step circular array beamforming is performed for each of the N circular sensor arrays 30, each array comprising M sensors 8, to produce N beam time series outputs. In a second step line array beamforming is performed on the N beam time series outputs of the first step producing one beam time series for the steered direction. The same process is then repeated for other beam steering directions of interest. Alternatively, the process is reversed performing line array beamforming first.

The decomposition process substantially facilitates cylindrical array beamforming. The number of mathematical operations and the amount of memory required to perform these operations are substantially reduced by expressing the cylindrical beamformer as a product of two sums, instead of a double summation as expressed by equation (3). This allows application of advanced beamforming processes for multidimensional arrays.

The circular and line array beamformers resulting from the decomposition process may be executed in parallel. This allows real time applications of an ultrasound imaging system using the architecture disclosed herein.

Decomposition processes for planar and spherical arrays are very similar to the decomposition process for the cylindrical array described above.

The beamforming process for line sensor arrays and circular sensor arrays 30, respectively, is a time delay beamforming estimator—basically a spatial filter. However, optimum beamforming relies upon beamforming filter coefficients chosen based on characteristics of noise received by the sensor array in order to optimize sensor array response. Processes for optimum beamforming using characteristics of noise received by the sensor array are called adaptive beamformers. Beamforming filter coefficients of these processes are chosen based on a covariance matrix of correlated noise received by the sensor array. However, if the knowledge of the noise's characteristic is inaccurate, performance of the adaptive beamformer will degrade significantly and may even result in cancellation of a desired signal. Therefore, it is very difficult to implement useful adaptive beamformers in real time operational systems. Furthermore, for post processing such as matched filter processing the adaptive beamformer has to provide coherent beam time series. In particular, the matched filter processing requires near-instantaneous convergence of the beamformer, producing a continuous beam time series correlating with a reference signal.

In adaptive beamforming, beamformer response is optimized to contain minimum contributions due to noise and signals arriving from directions other than a direction of a desired signal. For optimization, a linear filter vector Z is found, which is a solution to a constraint minimization problem that allows signals from a desired direction to pass with a specified gain. The solution of the minimization problem is expressed by

$$\overline{Z} ( {f}_{i} , θ ) = \frac{{R}^{- 1} ( {f}_{i} ) \overline{D} ( {f}_{i} , θ )}{{\overline{D}}^{*} ( {f}_{i} , θ ) {R}^{- 1} ( {f}_{i} ) \overline{D} ( {f}_{i} , θ )} . \qquad ( 6 )$$

D is the conventional steering vector. R(fᵢ) is a spatial correlation matrix of received sensor time series with elements Rₙₘ(f,δₙₘ)=E{Xₙ(f)Xₘ(f)}, wherein E{ . . . } denotes an expectation operator and δₙₘ is sensor spacing between n^{th} and m^{th} sensor.

Equation (6) provides adaptive steering vectors for beamforming signals received by a N-sensor array. In a frequency domain, an adaptive beam at a steering angle θₛ is then defined by

B(fᵢ,θₛ)=(Vector Z)*(fᵢ,θₛ)(vector X)(fᵢ), (7)

corresponding to conventional beams.

Thus output data from a conventional beamformer in a frequency domain is known and a corresponding output ξ(tᵢ,θₛ) in a time domain is then expressed as a weighted sum of steered sensor outputs

$$ξ ( {t}_{i} , {θ}_{s} ) = {\sum }_{n = 1}^{N} {x}_{n} ( {t}_{i} - {τ}_{s} ) . \qquad ( 8 )$$

Since ξ(tᵢ,θₛ) is an inverse fast Fourier transformation (IFFT) of B(f,θₛ), continuous beam time sequences are obtained from the output data of a frequency domain beamformer using fast Fourier transformation (FFT) and fast convolution processes.

Using the beamformer output data an expected broadband beam power B(θ) is determinable and is defined as the STCM in time domain and is assumed to be independent of tᵢ in stationary conditions. Supposing that Xₙ(fᵢ) is the Fourier transform of the sensor time series and assuming that the sensor time series are approximately band limited, a vector of steered sensor outputs xₙ(tᵢ,τₙ(θₛ)) is expressed by

$$\overline{x} ( {t}_{i} , {τ}_{n} ( {θ}_{s} ) ) = {{\sum }_{k = 1}}^{l + H} {T}_{k} ( {f}_{k} , {θ}_{s} ) \overline{X} ( {f}_{k} ) exp ( j 2 π {f}_{k} {t}_{i} ) . \qquad ( 11 )$$

T(fₖ,θ) is a diagonal steering matrix with elements identical to the elements of the conventional steering vector D and is expressed as follows:

$$T ( {f}_{k} , θ ) = [ \begin{matrix}1 & 0 & … & 0 \\ 0 & {d}_{1} ( {f}_{k} , θ ) & & \\ ⋮ & & ⋰ & ⋮ \\ 0 & & … & {d}_{N} ( {f}_{k} , θ )\end{matrix} ] . \qquad ( 12 )$$

The STCM follows then directly from the above equations as

$$Φ ( Δ f , {θ}_{s} ) = {{\sum }_{k = 1}}^{l + H} T ( {f}_{k} , {θ}_{s} ) R ( {f}_{k} ) {T}^{*} ( {f}_{k} , {θ}_{s} ) , \qquad ( 13 )$$

wherein the index k=1, 1+1, . . . , 1+H refers to frequency bins in a band of interest Δf and R(fₖ) is the CDSM for the frequency bin fₖ.

In steered minimum variance algorithms (STMV) a spectral estimate of broadband spatial power is generally used, however, estimates do not provide coherent beam time series because they represent the broadband beam power output of an adaptive process. Therefore, according to the invention the estimation process of the STMV has been modified to determine complex coefficients of Φ(Δf,θₛ) for all frequency bins in a frequency band of interest.

Accordingly, a STMV process is used in its original form to generate an estimate of Φ(Δf,θ) for all frequency bands Δf of a received signal. Assuming stationarity across the frequency bins of a band Δf the estimate of the STMV is considered to be approximately the same as a narrowband estimate Φ(f₀,θ) for a center frequency f₀ of the band Δf. Narrowband adaptive coefficients are then derived from

$$\overline{w} ( {f}_{0} , θ ) = \frac{{Φ ( {f}_{0} , Δ f , θ )}^{- 1} \overline{D} ( {f}_{0} , θ )}{{\overline{D}}^{*} ( {f}_{0} , θ ) {Φ ( {f}_{0} , Δ f , θ )}^{- 1} \overline{D} ( {f}_{0} , θ )} . \qquad ( 15 )$$

Phase variations across the frequency bins are modeled and using adaptive steering weights wₙ(Δf,θ) adaptive beams are formed.

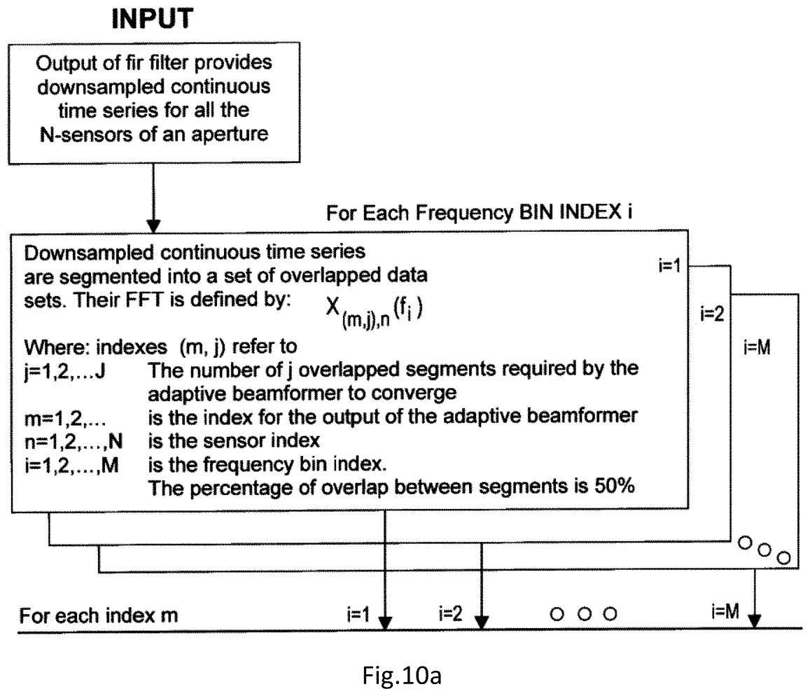

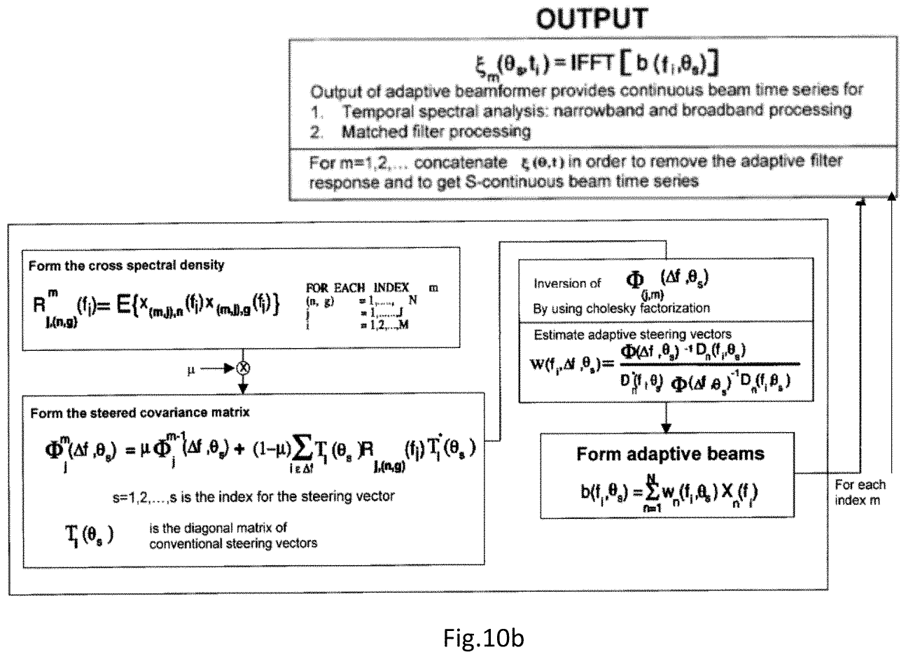

FIG. 10a and FIG. 10b show schematically the processing steps of the STMV beamformer as discussed above. First the sensor time series are segmented, overlapped and transformed into frequency domain using FFT. In a following step the STCM is determined. The STCM is then inverted using Cholesky factorization and in order to determine an estimation of the adaptive steering vectors. The adaptive steering vectors are then integrated in the frequency domain forming adaptive beams. Finally, adaptive beams in time domain are then formed through IFFT

b(tᵢ,θₛ,φₛ)=IFFT{B(fᵢ,θₛ,φₛ)},

wherein overlap and concatenation of segments are discarded to form a continuous beam time series.

Matrix inversion is a major issue for implementing adaptive beamformers in real time applications. Standard numerical methods for solving systems of linear equations can be applied to solve for the adaptive weights. The numerical methods include:
Cholesky factorization of the covariance matrix R(fₖ). This allows the linear system to be solved by backsubstitution in terms of the received data vector.

QR decomposition of the received vector X̅(fᵢ) which includes conversion of a matrix to upper triangular form via rotations. The QR decomposition method has better stability than the Cholesky factorization but requires twice as much computational effort.

SVD (Singular Value Decomposition) method. The SVD method is the most stable factorization technique but requires three time more computational effort than the QR decomposition method.

For investigative studies of the beamforming process Cholesky factorization and QR decomposition techniques have been applied. No noticeable differences in performance concerning stability have been found between these methods. Of course, for real time applications a fastest algorithm is often preferred.

Implementation of an adaptive beamformer with a large number of adaptive weights for a large number of sensors requires very long convergence periods eliminating dynamical characteristics of the adaptive beamformer to detect time varying characteristics of a received signal of interest. This limitation is avoided by reducing the number of adaptive weights. A reduction of the number of adaptive weights is achieved by introducing a sub-aperture processing scheme.

A sub-aperture configuration for a line array of sensors is described. The line array is divided into a plurality of overlapping sub-arrays. In a first stage the sub-arrays are beamformed using a conventional beamformer generating a number of sets of beams equal to the number of sub-arrays for each steering direction. In a second stage adaptive beamforming is performed on each set of beams steered in a same direction in space but each beam belonging to a different sub-array. A set of beams is equivalent to a line array consisting of directional sensors steered at a same direction with sensor spacing being equal to space separation between two contiguous sub-arrays and with the number of sensors being equal to the number of sub-arrays.

A second stage of beamforming comprises an adaptive beamformer on a line array consisting of, for example, G=3 beam time series b_{g}(tᵢ,θₛφₛ), g=1,2, . . . , G. For a given pair of azimuth and elevation steering angles {θₛ,φₛ}, the cylindrical adaptive beamforming process is reduced to an adaptive line array beamformer. The adaptive line array beamformer comprises only three beam time series b_{g}(tᵢθₛ,φₛ),g=1,2,3 with spacing δ=[(R2π/M)²+δ_{z}²]^{1/2} between two contiguous sub-aperture cylindrical cells, wherein (R2π/M) is the sensor spacing on each ring and wherein δ_{z} is the distance between each ring along the z-axis of the cylindrical array. The adaptive line array beamformer provides one or more adaptive beam time series with steering centered on the pair of azimuth and elevation steering angles {θₛ,φₛ}.

Because of the very small number of degrees of freedom in each sub-aperture the adaptation process experiences near-instantaneous convergence. Furthermore, the multidimensional sub-aperture beamforming process according to the invention may include a wide variety of adaptive noise cancellation techniques such as MVDR and GSC.

Furthermore, the sub-aperture configuration is applicable to other multidimensional arrays such as cylindrical arrays and spherical arrays. Decomposition, sub-aperture formation as well as implementation of adaptive beamformers for cylindrical and spherical arrays are similar to corresponding steps for planar arrays.

This decomposition process provides a foundation for an efficient signal processing implementation of the 3D Adaptive Beamformer and allows for a scalable and fully digital computing architecture design in accordance with the present embodiment.

The present embodiment includes a highly parallelized computing architecture for real time ultrasound imaging systems deploying 2D and/or 3D multidimensional ultrasound transducer array probes. The probes have planar, cylindrical or spherical geometrical sensor configurations. 3D adaptive signal processing flow and computing architecture layout of the present embodiment are applicable to 3D ultrasound imaging systems deploying either matrix (planar), cylindrical or spherical array ultrasound probes as are known. Alternatively, the probes have other configurations.

Referring to FIGS. 7a, 7b, and 7c, shown are simplified block diagrams of an implementation of an embodiment of the invention. Generic decomposition signal processing flow is shown in the diagrams. FIG. 7a as described hereinabove illustrates the data acquisition unit comprising a set of multiplexers for addressing each of the groups of sensors within the array. Furthermore, the A/DC peripherals are integrated with each one of the 32×32=1024 channels of the matrix (planar) array probe to digitize the transducer signals and to provide the resulting time series at the input of suitably programmed FPGAs to initiate image reconstruction processing. The D/AC peripherals are activated from the FPGAs to illuminate a medium of interest and to trigger a data acquisition process by the A/DC peripherals. The D/AC peripherals are for activating multi-focus illumination patterns, in the form of those shown in FIG. 5. These illumination patterns are unique in a sense that they illuminate the interior of the human body like a flashing light allowing the transducer array to record scattering signals from the illumination and to use the recorded signals within a volumetric image reconstruction process by the 3D Adaptive Beamformer. Referring to FIG. 7c shown is the architecture relying on many processes 791 such as that of FIG. 7b to result in a single image. This allows for quasi-real-time processing of 4D ultrasound image data.

Thus, the architecture of FIGS. 7a, 7b, and 7c for ultrasound imaging systems allows for a coherent processing for a set—in the embodiment {all}—of transducers of a deployed probe. Referring to FIG. 7b, shown is a simplified block diagram supporting the image processing process. Here data relating to an entire image 751 is provided for processing. The data is converted into a frequency domain with a fast Fourier transform (FFT) circuit 752. The converted data is then filtered 753, processed 754 and a reverse FFT is applied at 755 in order to form an image.

As detailed in FIG. 7d, the input information for the 3D adaptive beamformers include beam time series converted to a frequency domain of 3-snapshots from an output port of the conventional—time delay—beamformer as shown in FIG. 7b. A snapshot of data is defined as the planar array time series for supporting a complete volumetric image reconstruction process. This is important for the adaptive beamformer to achieve near instantaneous convergence. Alternatively, longer convergence is supported.

Relying on 3-snapshots by the 3D Adaptive beamformer, to achieve near instantaneous convergence, might be considered as an impediment in that it reduces the rate of the volumetric image reconstruction output images by a factor of 3. The current processing capacity of the computing architecture allows for the reconstruction of 20 volumes/second using the conventional time delay 3D beamformer. Thus, the time interval between two snapshots (i.e. two successive volumetric images) is 50.0 ms. As a result, the time interval between two successive volumetric image outputs of the 3D adaptive beamformer will be 150.0 ms.

This kind of impediment, however, is reduced by introducing, as detailed in FIG. 7d, a re-introduction or re-use of snapshot data to allow for an increase in the 3D Adaptive Beamforming output images to be the same as that of the conventional (time delay) beamformer. Thus, latency remains on the order of hundreds of milliseconds, but every 50 ms a new image is provided at an output port. Thus, and according to the acquisition arrangements of FIG. 7d, the time interval between two successive volumetric output images of the adaptive beamformer is 50.0 ms, except for an initial 100.0 ms of additional delay at the start. Alternatively, with faster processing circuitry, an image is provided at an output port of the beamformer every 12.5 ms with the adaptive beamformer re-using the previous 3 A-scans for each image reconstruction operation—performing one beamforming operation per MUX addressing operation. Alternatively, with enhanced processing speeds, enhanced resolution is supported instead of or in conjunction with increased frame rate.

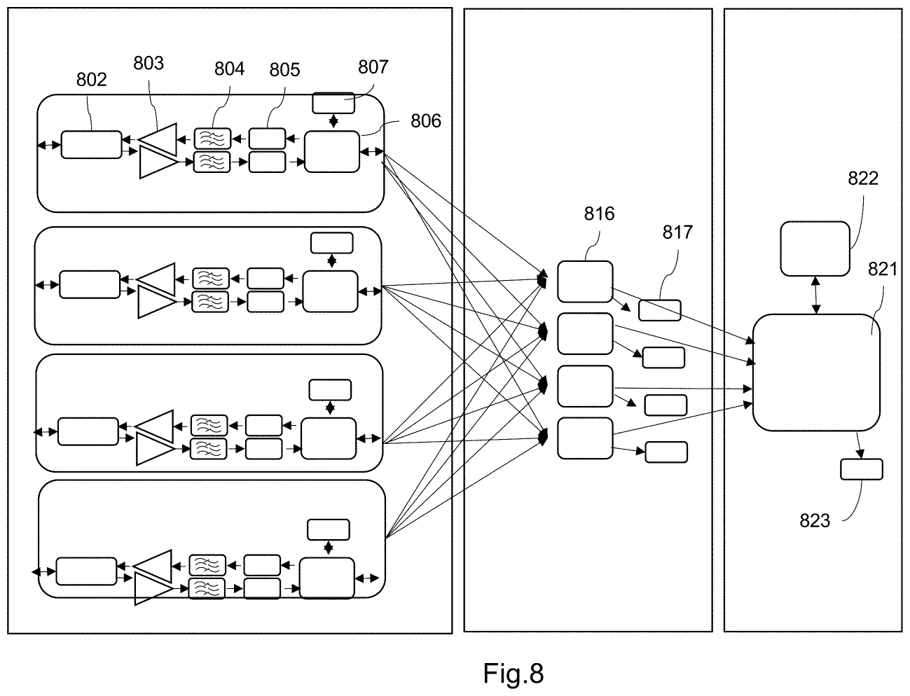

Referring to FIG. 8, shown is a data architecture diagram to highlight data flow during processing. As is evident from FIG. 8, between Csteer operations and Rsteer operations, there is a cross connect resulting in each stage communicating with all parallel subsequent stages. Thus, there is a limit to the amount of parallelization imposed by the communication between stages.

FIG. 8 depicts an innovative highly parallelized fully digital computing architecture for a real time 3D ultrasound imaging system that is capable of deploying a matrix array ultrasound probe with 32×32=1024-elements or 64×64-4096-elements or 16×16=256-elements.

The design principles of the computing architecture of FIG. 8 reflect the signal processing structure of the 3D Adaptive Beamformer of FIGS. 7c and 7d with elements including MUX 802, amplifier 803, filter 804, DAC/ADC 805, programmable logic 806 and memory 807 present in parallel implementations within the first stage; the first stage includes Csteer operations. In the 2^{nd} stage, programmable logic 816 and memory 817 support Rsteer implementation. In the third stage, a processor 821, a display 822, and a memory 823 allow for visualisation and storage. At the same time, the scalability and generic capabilities, for a variety of medical diagnostic applications, of the proposed computing architecture in FIG. 8, makes it a fully digital real time 3D ultrasound system incorporating unique illumination patterns and a complete digital 3D Adaptive Beamformer with fully coherent array gain incorporating, simultaneously, all transducer-elements of a deployed multidimensional array.

This highly parallelized architecture accommodates processing of from low to highly populated transducer arrays. For example, for a planar array probe with 16×16=256 transducers, the complexity of the computing architecture in FIG. 8, is reduced by a factor of 4 and results in deployment of a smaller number of FPGAs by a factor of 4, when compared to a planar array probe with 32×32=1024 transducers.

Numerous other embodiments may be envisaged without departing from the scope of the invention.

1. A method comprising:
firing a multidimensional array of ultrasound transducers comprising N transducers at a target;
sensing first reflected signals with at least some of the N transducers to produce first output signals;
providing the first output signals from only a first portion of the N transducers less than all the N transducers for storage and adaptive beamforming;
firing the multidimensional array of ultrasound transducers comprising N transducers at the target another time to produce second reflected signals;
sensing the second reflected signals with at least some of the N transducers to produce second output signals; and
providing the second output signals from only a second portion of the N transducers less than all the N transducers and different from the first portion for storage and adaptive beamforming.

2. The method of claim 1 wherein the first reflected signals are digitized to form the first output signals and wherein the second reflected signals are digitized to form the second output signals.

3. The method of claim 1 comprising:
performing adaptive beamforming on data derived from the first output signals and from the second output signals to determine a sensed topography; and
displaying an image of the sensed topography.

4. A method according to claim 3 wherein displaying the image data is performed in real time.

5. The method of claim 1 comprising:
firing the multidimensional array of ultrasound transducers comprising N transducers at the target another time;
sensing third reflected signals with at least some of the N transducers to produce third output signals; and
providing the third output signals from only a third portion of the N transducers less than all the N transducers and different from the first portion and different from the second portion for storage and adaptive beamforming.

6. The method of claim 5 wherein the first reflected signals are digitized to form the first output signals, wherein the second reflected signals are digitized to form the second output signals, and wherein the third reflected signals are digitized to form the third output signals.

7. The method of claim 6 comprising:
performing adaptive beamforming on data derived from the first output signals, from the second output signals, and from the third output signals to determine a sensed topography; and
displaying an image of the sensed topography.

8. The method of claim 5 wherein firing the multidimensional array of ultrasound transducers comprises forming a same illumination pattern with the multidimensional array of ultrasound transducers each time.

9. The method of claim 8 wherein the illumination pattern is conical.

10. The method of claim 8 wherein the illumination pattern is formed by beam steering and wherein each of the output signals used in adaptive beam forming for same output image data are captured relying upon a same illumination pattern.

11. The method of claim 1 wherein groups of the multidimensional array of ultrasound transducers are coupled via a multiplexer and addressed simultaneously, the multiplexing allowing the entire multidimensional array of ultrasound transducers to be read in n successive operations by incrementing the multiplexer addressing between operations.

12. The method of claim 11 wherein each multiplexer addresses four different ultrasound transducer elements of the multidimensional array of ultrasound transducers.

13. The method of claim 11 wherein adaptive beam forming is performed on an image comprising data from all of the transducer elements within the multidimensional array of ultrasound transducers.

14. The method of claim 13 wherein adaptive beamforming is performed in reliance upon two previously captured images, each of the previously captured images captured relying on a same illumination pattern.

15. The method of claim 14 wherein adaptive beam forming is performed in the frequency domain.

16. An ultrasound system comprising:
a multidimensional transducer array comprising a plurality N of ultrasound transducer elements arranged in an array, each of the plurality N of ultrasound transducers arranged for transmitting a beam steered signal together, and each of the plurality of transducers coupled to a multiplexer for switching between n of the plurality of ultrasound transducers such that there are at least N/n mulitplexers;
the multiplexers coupled for providing an information output signal from a selected one of the n transducers coupled therewith in response to a selection signal, each multiplexer for selecting between n transducers coupled therewith such that sampling of the information output signal from the N transducers is performed in n operations.

17. The ultrasound system of claim 16 wherein each of the plurality of ultrasound transducers is arranged for transmitting a steered signal simultaneously and only N/n ultrasound transducers are for being read simultaneously.

18. The ultrasound system of claim 16 wherein the multiplexers are each a 4:1 multiplexer.

19. The ultrasound system of claim 18 comprising an analog to digital converter coupled to an output port of each of the at least N/n mulitplexers.

20. A method comprising:
firing a multidimensional array of ultrasound transducers comprising N transducers at a target;
sensing first reflected signals with at least some of the N transducers to produce first output signals;
digitizing the first output signals to produce digitized first output signals;
transmitting some of the first output signals to a first processing circuit for performing Csteer processing;
transmitting others of the first output signals to a second other processing circuit for performing Csteer processing;
transmitting an output signal from the Csteer from each of the first processing circuit and the second processing circuit to a third processor for performing Rsteer processing; and
transmitting an output signal from the Csteer from each of the first processing circuit and the second processing circuit to a fourth other processor for performing Rsteer processing.
